(19) United States
(12) Patent Application Publication
(10) Pub. No.: US 2023/0033248 A1
(43) Pub. Date: Feb. 2, 2023

(54) COMPOSITIONS AND METHODS FOR ENHANCED DECONTAMINATION

(71) Applicant: Amir Reza Salar Parvini, San Diego, CA (US)

(72) Inventor: Amir Reza Salar Parvini, San Diego, CA (US)

(21) Appl. No.: 17/813,225

(22) Filed: Jul. 18, 2022

Related U.S. Application Data
(60) Provisional application No. 63/203,477, filed on Jul. 23, 2021.

(51) Int. Cl. A61K 8/60; A61K 8/19; C11D 3/02; C11D 3/22; A61K 8/42; A61K 8/73

(52) U.S. Cl. CPC A61K 8/602; A61K 8/19; C11D 3/02; C11D 3/225; A61K 8/42; A61K 8/731; A61K 2800/41

(57) ABSTRACT

Provided herein are compositions for removing and/or suppressing a hazardous material from an environment or from a body after an activity in athletic and recreational facilities. The hazardous material includes dusts or contaminants, including per- and poly-fluoroalkyl substances (PFAS), polycyclic aromatic hydrocarbons (PAH), and heavy metals. Also disclosed are methods of preparing the compositions and methods for decontamination of a surface with the compositions.

### CROSS-REFERENCE TO RELATED APPLICATIONS

This application is claims priority to U.S. Provisional Patent Application No. 63/203,477 filed Jul. 23, 2021, which is hereby incorporated by reference in its entirety.

### FIELD

The present disclosure generally relates to compositions for removing and/or suppressing hazardous materials, including dusts and contaminants from an environment or from a surface. More particularly, the present disclosure relates to compositions and methods of removing and/or suppressing dusts, per- and poly-fluoroalkyl substances (PFAS), polycyclic aromatic hydrocarbons (PAH), and/or heavy metals from an environment or from a surface. The compositions and methods may be used for dust mitigation. Methods for preparing the compositions are also provided.

### BACKGROUND

Athletic and recreational facilities (including, for example, artificial turf and children's playground infill) contain hazardous substances, including dusts, per- and poly-fluoroalkyl substances (PFAS), polycyclic aromatic hydrocarbons (PAH), and/or heavy metals. These hazardous substances are also present in firefighting chemicals and other industrial settings and can be transferred to skin or clothing, creating undesirable exposure to the hazardous substances. Upon exposure, airways and human skin absorbs significant amounts of these the hazardous substances: 50-70% of PFAS; 20-56% of PAHs; and 0.1-2% of the heavy metals. Failure to reduce exposure of these substance and/or to remove these hazardous substances from the skin with an effective cleanser within 30 minutes of exposure allows the substances to be absorbed dermally and enter the body where they can remain absorbed long-term and are capable of causing numerous negative health risks and/or issues. Currently there are no specific solutions for athletes, children, and other individuals who are exposed to these hazardous compounds to reduce exposure to and reduce the dermal absorption of dusts, PFAS, PAH, and/or heavy metals after an activity that includes contact with athletic and recreational facilities.

The need exists for a composition that can be applied to an environment to remediate, remove, and/or suppress these hazardous materials in the environment, or that can be applied topically to reduce the dermal absorption of dusts, PFAS, PAH, and/or heavy metals by the skin of individuals exposed to such substances.

### SUMMARY

The present disclosure describes compositions having a surfactant, a polymer, an adsorbent, and a chelator. Also described are methods of making and using the compositions. The compositions and methods of making and using the compositions described herein can be used to remove and/or suppress hazardous materials, including dusts and contaminants from an environment or from a surface.

Accordingly, provided herein are compositions that include a surfactant, a polymer, an adsorbent, and a chelator. In some embodiments, the surfactant includes a glycoside, a cocoamphodiacetate, or a combination thereof. In some embodiments, the glycoside includes a C₆₋₃₀ alkyl glycoside. In some embodiments, the glycoside includes decyl glucoside. In some embodiments, the cocoamphodiacetate includes disodium cocoamphodiacetate. In some embodiments, the polymer includes cellulose. In some embodiments, the cellulose includes a cellulose derivative. In some embodiments, the cellulose includes carboxymethylcellulose. In some embodiments, the adsorbent includes charcoal, activated carbon, granular activated carbon (GAC), clay minerals, bentonite, sand, or a combination thereof, optionally wherein the adsorbent is protonated. In some embodiments, the adsorbent is protonated. In some embodiments, the adsorbent includes protonated activated carbon (PAC). In some embodiments, the adsorbent is a particle having a diameter of 0.2 mm. In some embodiments, the chelator includes at least one heavy metal chelator. In some embodiments, the chelator includes an ethylenediamine, a gluconate, a polycarboxylic acid, or a combination thereof. In some embodiments, the surfactant is present in an amount ranging from about 0.001 to about 50 weight percent. In some embodiments, the polymer is present in an amount ranging from about 0.001 to about 10 weight percent. In some embodiments, the adsorbent is present in an amount ranging from about 0.0001 to about 50 weight percent.

Some embodiments provided herein relate to compositions that include decyl glucoside, disodium cocoamphodiacetate, carboxymethylcellulose, trisodium ethylenediamine disuccinate, sodium gluconate, citric acid, sodium benzoate, potassium sorbate, protonated activated carbon, and water. In some embodiments, the decyl glucoside is present in an amount ranging from about 0.001 to about 50 weight percent. In some embodiments, the disodium cocoamphodiacetate is present in an amount ranging from about 0.001 to about 10 weight percent. In some embodiments, the carboxymethylcellulose is present in an amount ranging from about 0.001 to about 10 weight percent. In some embodiments, the trisodium ethylenediamine disuccinate is present in an amount ranging from about 0.001 to about 10 weight percent. In some embodiments, the sodium gluconate is present in an amount ranging from about 0.001 to about 10 weight percent. In some embodiments, the citric acid is present in an amount ranging from about 0.001 to about 10 weight percent. In some embodiments, the sodium benzoate is present in an amount ranging from about 0.001 to about 10 weight percent. In some embodiments, the potassium sorbate is present in an amount ranging from about 0.001 to about 10 weight percent. In some embodiments, the protonated activated carbon is present in an amount ranging from about 0.0001 to about 10 weight percent. In some embodiments, the composition includes decyl glucoside present in an amount of about 16 weight percent, disodium cocoamphodiacetate present in an amount of about 5 weight percent, carboxymethylcellulose present in an amount of about 0.99 weight percent, trisodium ethylenediamine disuccinate present in an amount of about 0.2 weight percent, sodium gluconate present in an amount of about 0.2 weight percent, citric acid present in an amount of about 1.1 weight percent, sodium benzoate present in an amount of about 0.05 weight percent, potassium sorbate present in an amount of about 0.05 weight percent, protonated activated carbon present in an amount of about 0.05 weight percent, and water in balance. In some embodiments, the pH of the composition is in the range from about 3 to about 8. In some embodiments, the composition is configured to adsorb at least one metal. In some embodiments, the density of the metal is equal to or greater than about 5 kg/m³. In some embodiments, the metal is selected from Al, As, Ba, Be, Cd, Co, Cr, Cu, Hg, Fe, Li, Mg, Mn, Mo, Ni, Pb, Rb, Sb, Se, Sn, Sr, Tl, V, W, and Zn. In some embodiments, the composition is configured to adsorb, remove, remediate, and/or suppress hazardous materials, including dusts or other contaminants. In some embodiments, the hazardous materials include dusts, perfluoroalkyl substances, polyfluoroalkyl substances, polycyclic aromatic hydrocarbons, heavy metals, or a combination thereof. In some embodiments, the composition is formulated as a personal care product. In some embodiments, the personal care product is a cleanser, a soap, a salve, a scrub, a wash, a cream, a hair conditioner, a lotion, a shampoo, a wipe, or a combination thereof.

Some embodiments provided herein relate to methods of removing, remediating, adsorbing, and/or suppressing hazardous materials, including dusts or contaminants. In some embodiments, the methods include applying a composition to a material having or suspected of having a hazardous material thereon, absorbing and/or suppressing hazardous materials from the material to form a composition with absorbed contaminants, and removing or suppressing the composition with absorbed contaminants from the material. In some embodiments, the contaminants include perfluoroalkyl substances, polyfluoroalkyl substances, polycyclic aromatic hydrocarbons, heavy metals, or a combination thereof. In some embodiments, the compositions include any of the compositions disclosed herein. In some embodiments, the material is a surface. In some embodiments, the surface comprises skin of a subject. In some embodiments, the subject is a mammal. In some embodiments, the subject is human. In some embodiments, the heavy metal is selected from Al, As, Ba, Be, Cd, Co, Cr, Cu, Hg, Fe, Li, Mg, Mn, Mo, Ni, Pb, Rb, Sb, Se, Sn, Sr, Tl, V, W, and Zn.

### DETAILED DESCRIPTION

Embodiments of the present disclosure relate to compositions for removing and/or suppressing contaminants present in any environment contaminants, and compositions for application to a surface. Contaminants may include any hazardous substance capable of being breathed in the airway, including in the lungs and/or absorbed through the skin, including, for example, dusts, per- and poly-fluoroalkyl substances (PFAS), polycyclic aromatic hydrocarbons (PAH), and heavy metals. Methods for making and using the compositions are also disclosed herein.

The following description provides context and examples but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

It will be readily understood that the aspects of the present disclosure, as generally described herein, can be arranged, substituted, combined, separated, and designed in a wide variety of different configurations, all of which are explicitly contemplated herein.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose.

### Compositions

Some embodiments provided herein relate to compositions for removal and/or suppression of a contaminant from an environment or from a surface, and/or for dust mitigation. In some embodiments, the compositions include one or more of a surfactant, a polymer, an adsorbent, or a chelator, or any combination thereof. In some embodiments, the compositions include all of the aforementioned components, including one or more surfactant, one or more polymer, one or more adsorbent, and one or more chelator. In some embodiments, the compositions include any one of the aforementioned components, any two of the aforementioned components, any three of the aforementioned components, or all four of the aforementioned components. In some embodiments, the compositions include one or more of each component, alone or in combination with one or more of each other component. The selection of which components and how many of the aforementioned components will vary depending on the specific application and/or the specific product formulation.

As used herein, the term “environment” has its ordinary meaning as understood in light of the specification and refers to an area in which a contamination may be located, or in which a contaminant is likely to be found. For example, an environment may include a playground infill, a turf infill, or any location in which artificial turf is located. Other environments may include industrial settings, or any location in which hazardous contaminants may be found.

As used herein, the term “surface” has its ordinary meaning as understood in light of the specification and refers to an outer facing boundary of an object. A surface can include an artificial surface, such as a surface of a playground, a surface of a field, a surface of an artificial turf blade, a surface of clothing, a surface of equipment, a surface of furniture, or a surface of any artificial material. A surface can include a biological surface, such as skin of a subject.

In some embodiments, the compositions include a surfactant. As used herein, the term “surfactant” has its ordinary meaning as understood in light of the specification and refers to a surface active agent which has both hydrophilic and hydrophobic parts. In some embodiments, the surfactant includes a nonionic surfactant, including, for example, an ethoxylate or a glucoside. In some embodiments, the surfactant includes a zwitterionic surfactant, such as a phospholipid or disodium cocoamphodiacetate. In some embodiments, the surfactant includes a glycoside, a cocoamphodiacetate, or any combination thereof.

In some embodiments, the compositions include at least one surfactant. In some embodiments, the compositions include two or more surfactants. In some embodiments, the surfactant includes wetting agents, emulsifiers, foaming agents, dispersants, and any combination of the foregoing. In some embodiments, the surfactant includes a surfactant that is amphiphilic, amphoteric, anionic, cationic, zwitterionic, non-ionic, and any combination of the foregoing. In some embodiments, the surfactant can be obtained from an agricultural product. In some embodiments, the surfactant can be obtained from coconut trees or a product thereof, such as coconut husks.

In some embodiments, the surfactant includes a glycoside. In some embodiments, the surfactant includes an O-glycoside (an O-glycosyl compound), a glycosylamine (an N-glycosyl compound), a thioglycoside (an S-glycosyl compound), a C-glycoside (a C-glycosyl compound), and any combination of the foregoing. In some embodiments, the surfactant includes a C₆₋₃₀ alkyl glucoside, a C₆₋₃₀ alkyl fructoside, a C₆₋₃₀ alkyl glucuronide, and any combination of the foregoing. In some embodiments, the surfactant includes a C₆₋₁₈ or C₆₋₃₀ alkyl glucoside. In some embodiments, the surfactant includes hexyl glucoside, octyl glucoside, decyl glucoside, dodecyl glucoside, tetradecyl glucoside, hexadecyl glucoside, octadecyl glucoside, and any combination of the foregoing. In some embodiments, the surfactant includes decyl glucoside.

In some embodiments, the surfactant includes a cocoamphodiacetate. In some embodiments, the surfactant includes cocoamphodiacetate having cationic counterions. In some embodiments, the cationic counterions include cations of metals, inorganic compounds, organic compounds, and any combination of the foregoing. In some embodiments, the cationic counterions include cations of alkali metals, alkaline earth metals, and any combination of the foregoing. In some embodiments, the surfactant includes potassium cocoamphodiacetate, sodium cocoamphodiacetate, magnesium cocoamphodiacetate, calcium cocoamphodiacetate, dipotassium cocoamphodiacetate, disodium cocoamphodiacetate, and any combination of the foregoing. In some embodiments, the surfactant includes disodium cocoamphodiacetate.

In some embodiments, the surfactant includes decyl glucoside and disodium cocoamphodiacetate.

In some embodiments, the surfactant is present in the compositions at a weight percent of equal to or less than about 0.001 to about 60 weight percent, such as 0.001%, 0.005%, 0.01%, 0.05%, 0.1%, 0.5%, 1%, 2%, 3%, 4%, 5%, 6%, 7%, 10%, 12%, 13%, 14%, 15%, 16%, 17%, 18%, 19%, 20%, 25%, 30%, 40%, 50%, 60%, or in an amount within a range defined by any of the aforementioned values.

In some embodiments, the compositions include a polymer. As used herein, the term “polymer” has its ordinary meaning as understood in light of the specification and refers to prepolymers, oligomers and both homopolymers and copolymers. In some embodiments, the polymer acts as a gelling agent. As used herein, a “gelling agent” has its ordinary meaning as understood in light of the specification and refers to an agent that modulates the viscosity of a composition.

In some embodiments, the polymer includes cellulose, a cellulose derivative, and any combination of the foregoing. In some embodiments, the polymer includes a cellulose ether, a cellulose ester, and any combination of the foregoing. In some embodiments, the polymer includes a cellulose alkyl ether, a cellulose hydroxyalkyl ether, a cellulose carboxyalkyl ether, and any combination of the foregoing. In some embodiments, the polymer includes methylcellulose, ethylcellulose, ethyl methylcellulose, methylcellulose, hydroxyethyl cellulose, hydroxypropyl cellulose, hydroxyethyl methyl cellulose, hydroxypropyl methyl cellulose, ethyl hydroxyethyl cellulose, carboxymethylcellulose carboxyethylcellulose, and any combination of the foregoing. In some embodiments, the polymer includes carboxymethylcellulose.

In some embodiments, the polymer is present in the compositions at a weight percent of equal to or less than about 0.001 to about 40 weight percent, such as 0.001%, 0.005%, 0.01%, 0.05%, 0.1%, 0.2%, 0.3%, 0.4%, 0.5% 0.6%, 0.7%, 0.8%, 0.9%, 1%, 1.01%, 1.05%, 1.1%, 1.2%, 1.3%, 1.4%, 1.5%, 2%, 2.5%, 3%, 5%, 10%, 15%, 20%, 25%, 30%, 40%, or in an amount within a range defined by any of the aforementioned values.

In some embodiments, the compositions include an adsorbent. As used herein, the term “adsorbent” has its ordinary meaning as understood in light of the specification and refers to a material in which atoms, ions, biomolecules, or molecules of gas, liquid, or dissolved solids adhere. For example, an adsorbent is a material upon which a contaminant adheres.

In some embodiments, the adsorbent includes activated carbon. In some embodiments, the adsorbent includes activated carbon obtained from an agricultural product. In some embodiments, the adsorbent includes activated carbon obtained from coconut trees or a product thereof, such as coconut husks. In some embodiments, the adsorbent includes granular activated carbon (GAC). In some embodiments, the adsorbent is protonated. In some embodiments, the adsorbent includes protonated activated carbon (PAC). In some embodiments, the adsorbent includes protonated granular activated carbon (PGAC). In some embodiments, a particle size of the adsorbent is equal to or less than about 0.2 millimeters (mm). In some embodiments, the particle size of the adsorbent is about 0.01, 0.02, 0.03, 0.04, 0.06, 0.08, 0.1, 0.12, 0.14, 0.16, 0.18, 0.2, 0.25, 0.3, 0.4, 0.6 mm, or a size within a range defined by any of the aforementioned values. Without wishing to be bound by theory, in some embodiments, protonated carbon acts as an adsorption site for PFAS, allowing for binding and rinse off of PFAS. In some embodiments, the protonated adsorbent increased efficacy of chelation of PFAS by a factor two to eight fold. In some embodiments, protonated adsorbents remove long chain PFAS in an amount of more than 40%, more than 50%, more than 60%, more than 70%, more than 80%, more than 90%, more than 95%, or more than 99%.

In some embodiments, the adsorbent is present in the compositions at a weight percent of equal to or less than about 0.0001 to about 50 weight percent, such as 0.0001%, 0.0005%, 0.001%, 0.005%, 0.01%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 8%, 10%, 15%, 20%, 30%, 40%, or 50%, or in an amount within a range defined by any of the aforementioned values.

In some embodiments, the compositions include a chelator. As used herein, the term “chelator” has its ordinary meaning as understood in light of the specification and refers to an inorganic or organic chemical compound that binds another chemical species, such as metal ions or atoms. In some embodiments, the chelator suppresses chemical activity of the other chemical species. In some embodiments, the chelator includes at least one heavy metal chelator. The term “heavy metal chelator,” as used herein, is an inorganic or organic chemical compound that bonds a heavy metal. The term “heavy metal,” as used herein, is an element having one or more of the following properties: a density equal to or greater than about 5 kg/m³; toxicity at concentrations above about 10 parts per million; classified as a metalloid or metal on the Periodic Table of Elements; and one of the elements Al, As, Ba, Be, Bi, Cd, Co, Cr, Cu, Hg, Fe, Li, Mg, Mn, Mo, Ni, Pb, Rb, Sb, Se, Sn, Sr, Tl, V, W, and Zn.

In some embodiments, the chelator includes at least one heavy metal chelator. In some embodiments, the compositions include at least one chelator or heavy metal chelator. In some embodiments, the compositions include any combination of two or more chelators or heavy metal chelators. In some embodiments, the compositions include any combination of three chelators or heavy metal chelators. In some embodiments, the chelator or heavy metal chelator includes an inorganic or organic chemical compound, derivatized chitosan, plant material, fumed silica, and any combination of the foregoing. In some embodiments, the chelator or heavy metal chelator includes a biodegradable chemical compound.

In some embodiments, the chelator or heavy metal chelator includes a chemical compound selected from a crown ether, an ethylenediaminetetraacetate (EDTA), a gluconate, an oxalate salt, a polycarboxylic acid, a polyaminopolycarboxylic acid, and any combination of the foregoing. In some embodiments, the chelator or heavy metal chelator includes an EDTA, a gluconate, a polycarboxylic acid, and any combination of the foregoing.

In some embodiments, the chelator or heavy metal chelator includes trisodium ethylenediamine disuccinate; calcium disodium EDTA; a disodium, trisodium, tetrasodium, dipotassium, tripotassium, dilithium or diammonium salt of EDTA; a barium, calcium, cobalt, copper, dysprosium, europium, iron, indium, lanthanum, magnesium, manganese, nickel, samarium, strontium, or zinc chelate of EDTA. In some embodiments, the chelator or heavy metal chelator includes trisodium ethylenediamine disuccinate. In some embodiments, the trisodium ethylenediamine disuccinate is a powder.

In some embodiments, the chelator or heavy metal chelator includes an oxalate salt such as, for example, sodium oxalate salt, potassium oxalate salt, ammonium oxalate salt, lithium oxalate salt, and any combination of the foregoing.

In some embodiments, the chelator or heavy metal chelator includes a gluconate salt. In some embodiments, the gluconate salt includes cations of metals, inorganic compounds, organic compounds, and any combination of the foregoing. In some embodiments, the gluconate salt includes cations of alkali metals, alkaline earth metals, and any combination of the foregoing. In some embodiments, the chelator or heavy metal chelator includes potassium gluconate, sodium gluconate, and any combination of the foregoing. In some embodiments, the chelator or heavy metal chelator includes sodium gluconate.

In some embodiments, the chelator or heavy metal chelator includes a polycarboxylic acid. In some embodiments, the chelator or heavy metal chelator includes a dicarboxylic acid, a tricarboxylic acid, and any combination of the foregoing. In some embodiments, the chelator or heavy metal chelator includes citric acid, tartaric acid, oxalic acid, and any combination of the foregoing. In some embodiments, the chelator or heavy metal chelator includes citric acid.

In some embodiments, the chelator or heavy metal chelator is selected from the group consisting of 2,3-dimercaptopropanesulphonate sodium (2,3-dimercato-1-propanesulfonic acid, or DMP S), 2,3-dimercaptosuccinic acid (DMSA), British anti-Lewisite (BAL), ethyleneglycol-bis[beta-aminoethyl ether]-N,N′-tetra-acetic acid (EGTA), diethylenetriamine-pentaacetic acid (DTPA), dipropylene-triamine (DPTA), triethylenetetraaminehexaacetic acid (TTHA), N-hydroxy ethylene-diaminehexaacetic-acid (HEDHA), 1,4,7-triazacyclononane-N,N′,N″-triacetic acid (NOTA), 1,4,7,10-tetraazacyclododecane-N,N′,N″,N′″-tetraacetic acid (DOTA), hydroxyethyl-ethylenediaminetriacetic acid (HEDTA, e.g., an N-(2-hydroxyethyl)-ethylenediaminetriacetic acid), iminodiacetic acid (IDA), penicillamine, dimercaptosuccinic acid, citrate, tartrate, thiomalic acid, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), and 2,2′-dipyridyl.

In some embodiments, the chelator or heavy metal chelator includes trisodium ethylenediamine disuccinate, sodium gluconate, and citric acid.

In some embodiments, the chelator is present in the compositions at a weight percent of equal to or less than about 0.001 to about 50 weight percent, such as 0.001%, 0.005%, 0.01%, 0.05%, 0.1%, 0.15%, 0.16%, 0.17%, 0.18%, 0.19%, 0.2%, 0.21%, 0.22%, 0.23%, 0.24%, 0.25%, 0.3%, 0.4%, 0.5%, 0.6%, 0.7%, 0.8%, 0.9%, 1%, 1.1%, 1.2%, 1.3%, 1.4%, 1.5%, 2%, 3%, 4%, 5%, 10%, 15%, 20%, 25%, 30%, 40% or 50%, or in an amount within a range defined by any of the aforementioned values.

In some embodiments, the compositions include a buffer. In some embodiments, the buffer includes a polycarboxylic acid, a polycarboxylate, or a combination thereof. In some embodiments, the buffer includes a dicarboxylic acid, dicarboxylate, a tricarboxylic acid, a tricarboxylate, and any combination of the foregoing. In some embodiments, the buffer includes acetic acid, acetate, boric acid, borate, phosphoric acid, phosphonate, citric acid, citrate, tartaric acid, tartrate, oxalic acid, oxalate, and any combination of the foregoing. In some embodiments, buffer includes a citric acid. In some embodiments, buffer includes a citrate. In some embodiments, the buffer is present in the compositions at a weight percent of equal to or less than about 0.01 to about 40 weight percent, such as 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or in an amount within a range defined by any of the aforementioned values.

In some embodiments, the compositions include at least one preservative. Any preservative now known or yet to be discovered may be used in the compositions disclosed herein. In some embodiments, the preservative includes benzyl alcohol, methyl paraben, propyl paraben, DMDM hydantoin, methylchloroisothiaoline, methylisothiazolinone, imidazolidinyl urea phenoxyethanol, potassium sorbate, sodium benzoate, benzoic acid, phenoxyethanol, propyl paraben, methyl paraben, benzalkonium chloride, poly(hexamethylenebiguanide) hydrochloride, or any combination thereof.

In some embodiments, the preservative includes potassium sorbate, sodium benzoate, or a combination thereof. In some embodiments, the preservative includes potassium sorbate. In some embodiments, the preservative includes sodium benzoate.

In some embodiments, the preservative is present in the compositions, either alone or in combination, at a weight percent of equal to or less than about 0.001 to about 10 weight percent, such as 0.001%, 0.005%, 0.01%, 0.02%, 0.03%, 0.04%, 0.045%, 0.05%, 0.055%, 0.06%, 0.065%, 0.07%, 0.075%, 0.08%, 0.09%, 0.1%, 0.2%, 0.5%, 1%, 2%, 3%, 5%, 10%, or in an amount within a range defined by any of the aforementioned values.

In some embodiments, the compositions include at least one humectant or dispersant. In some embodiments, the humectant or dispersant includes an essential oil, a glycerin, a glycol, a colloid, an agar, a carbomer, a hydroscopic substituted cellulose, a starch, a propylene glycol glycerin, sorbitol, and any combination of the foregoing. A non-limiting example of a glycerin suitable for use includes vegetable glycerin. Non-limiting examples of polyglycols suitable for use include polyethylene glycol and polypropylene glycol. In some embodiments, the humectant or dispersant includes vegetable glycerin, at least one essential oil, or a combination thereof. In some embodiments, the humectant or dispersant includes vegetable glycerin. In some embodiments, the humectant or dispersant includes at least one essential oil.

In some embodiments, the humectant or dispersant is present in the compositions at a weight percent of equal to or less than about 0.01 to 40 weight percent, such as 0.01%, 0.05%, 0.1%, 0.5%, 2%, 3%, 3.2%, 3.4%, 3.5%, 3.6%, 3.7%, 3.8%, 3.9%, 4%, 4.1%, 4.2%, 4.3%, 4.4%, 4.5%, 5%, 6%, 7%, 10%, 12%, 13%, 14%, 15%, 16%, 17%, 18%, 19%, 20%, 25%, 30%, 40%, or in an amount within a range defined by any of the aforementioned values.

In some embodiments, the compositions include at least one solvent. The solvent is a carrier for the combination of components and is one in which the combination of components are soluble. In some embodiments, the solvent includes water, at least one polyol, such as, for example, propylene glycol, butylene glycol, glycerol, and any combination of the foregoing. Non-limiting examples of the polyol include propylene glycol, butylene glycol, and glycerol. In some embodiments, the solvent includes water. Non-limiting examples of water suitable for use include deionized water, distilled water, filtered water, tap water, spring water, glacier water, or natural water.

In some embodiments, the solvent is present in the compositions at a weight percent of equal to or less than about 0.1%, 0.5%, 1%, 5%, 10%, 15%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 85%, 90%, 95%, 98%, 99%, or ranges including and/or spanning the aforementioned values.

Some embodiments of the present disclosure relate to compositions that include one or more of decyl glucoside, disodium cocoamphodiacetate, carboxymethylcellulose, trisodium ethylenediamine disuccinate, sodium gluconate, citric acid, sodium benzoate, potassium sorbate, or protonated activated carbon, and any combination of the aforementioned components. In some embodiments, the compositions include all of the aforementioned components, including decyl glucoside, disodium cocoamphodiacetate, carboxymethylcellulose, trisodium ethylenediamine disuccinate, sodium gluconate, citric acid, sodium benzoate, potassium sorbate, and protonated activated carbon. In some embodiments, the compositions include any one of the aforementioned components, any two of the aforementioned components, any three of the aforementioned components, any four of the aforementioned components, any five of the aforementioned components, any six of the aforementioned components, any seven of the aforementioned components, any eight of the aforementioned components, or all nine of the aforementioned components. The selection of which components and how many of the aforementioned components will vary depending on the specific application and/or the specific product formulation.

In some embodiments, decyl glucoside is present in the compositions at a weight percent of equal to or less than about 0.1%, 0.5%, 2%, 3%, 4%, 5%, 6%, 7%, 10%, 12%, 13%, 14%, 15%, 16%, 17%, 18%, 19%, 20%, 25%, 30%, 40%, 50%, 60%, or ranges including and/or spanning the aforementioned values. In some embodiments, disodium cocoamphodiacetate is present in the compositions at a weight percent of equal to or less than about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or ranges including and/or spanning the aforementioned values. In some embodiments, carboxymethylcellulose is present in the compositions at a weight percent of equal to or less than about 0.01%, 0.05%, 0.1%, 0.2%, 0.6%, 0.7%, 0.8%, 0.9%, 0.95%, 0.98%, 0.99%, 1%, 1.01%, 1.02%, 1.03%, 1.04%, 1.05%, 1.06%, 1.1%, 1.2%, 1.3%, 1.6%, 2%, 2.5%, 3%, 5%, 10%, 15%, 20%, 25%, 30%, 40%, or ranges including and/or spanning the aforementioned values. In some embodiments, trisodium ethylenediamine disuccinate is present in the compositions at a weight percent of equal to or less than about 0.001%, 0.005%, 0.01%, 0.05%, 0.1%, 0.15%, 0.16%, 0.17%, 0.18%, 0.19%, 0.2%, 0.21%, 0.22%, 0.23%, 0.24%, 0.25%, 0.3%, 0.4%, 0.5%, 0.6%, 0.75%, 0.9%, 0.95%, 1%, 2%, 3%, 4%, 5%, 10%, 15%, 20%, 25%, 30%, or ranges including and/or spanning the aforementioned values. In some embodiments, sodium gluconate is present in the compositions at a weight percent of equal to or less than about 0.001%, 0.005%, 0.01%, 0.05%, 0.1%, 0.15%, 0.16%, 0.17%, 0.18%, 0.19%, 0.2%, 0.21%, 0.22%, 0.23%, 0.24%, 0.25%, 0.3%, 0.4%, 0.5%, 0.6%, 0.75%, 0.9%, 0.95%, 1%, 2%, 3%, 4%, 5%, 10%, 15%, 20%, 25%, 30%, or ranges including and/or spanning the aforementioned values. In some embodiments, citric acid is present in the compositions at a weight percent of equal to or less than about 0.001%, 0.005%, 0.01%, 0.05%, 0.1%, 0.2%, 0.3%, 0.4%, 0.5%, 0.6%, 0.75%, 0.9%, 0.95%, 0.96%, 0.97%, 0.98%, 0.99%, 1%, 1.05%, 1.06%, 1.07%, 1.08%, 1.09%, 1.1%, 1.11%, 1.12%, 1.13%. 1.14%, 1.15%, 1.2%, 1.3%, 1.4%, 1.5%, 1.8%, 2%, 3%, 4%, 5%, 10%, 15%, 20%, 25%, 30%, or ranges including and/or spanning the aforementioned values. In some embodiments, sodium benzoate is present in the compositions at a weight percent of equal to or less than about 0.001%, 0.005%, 0.01%, 0.02%, 0.03%, 0.04%, 0.045%, 0.05%, 0.055%, 0.06%, 0.065%, 0.07%, 0.075%, 0.08%, 0.09%, 0.1%, 0.2%, 0.5%, 1%, 2%, 3%, 5%, 10%, or ranges including and/or spanning the aforementioned values. In some embodiments, potassium sorbate is present in the compositions at a weight percent of equal to or less than about 0.001%, 0.005%, 0.01%, 0.02%, 0.03%, 0.04%, 0.045%, 0.05%, 0.055%, 0.06%, 0.065%, 0.07%, 0.075%, 0.08%, 0.09%, 0.1%, 0.2%, 0.5%, 1%, 2%, 3%, 5%, 10%, or ranges including and/or spanning the aforementioned values. In some embodiments, protonated activated carbon is present in the compositions at a weight percent of equal to or less than about 0.001%, 0.005%, 0.01%, 0.02%, 0.03%, 0.04%, 0.045%, 0.05%, 0.055%, 0.06%, 0.065%, 0.07%, 0.075%, 0.08%, 0.09%, 0.1%, 0.2%, 0.5%, 1%, 2%, 3%, 5%, 10%, or ranges including and/or spanning the aforementioned values. In some embodiments, water is present in the compositions at a weight percent of equal to or less than about 0.1%, 0.5%, 1%, 5%, 10%, 15%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 85%, 90%, 95%, 98%, 99%, or ranges including and/or spanning the aforementioned values.

The compositions disclosed herein can be acidic, alkaline, or neutral. In some embodiments, the compositions are acidic. In some embodiments, the compositions are alkaline. In some embodiments, a pH of the compositions ranges from about 3 to about 8, such as 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6, 6.5, 7, 7.5, or 8 or ranges including and/or spanning the aforementioned values.

In some embodiments, the compositions are formulated to reduce, suppress, adsorb, and/or remove at least one contaminant. As used herein, the term “contaminant” has its ordinary meaning as understood in light of the specification and refers to a substance or substances that are hazardous to health, that are not intended to be present in a certain environment or on a certain surface, and/or that are desirable to be removed from an environment or from a surface. Examples of contaminants include, for example, dusts, per- and poly-fluoroalkyl substances (PFAS), polycyclic aromatic hydrocarbons (PAHs), and/or heavy metals.

In some embodiments, the contaminant includes dusts. As used herein, “dusts” has its ordinary meaning as understood in light of the specification, and refers to fine particles of solid matter that can be from various sources, including from soil, from artificial environments (such as artificial turfs), pollens, minerals, or other environments. Dusts may cause various problems, including with breathing, health, irritation, vision, or environmental contamination. In some embodiments, the compositions described herein are used for dust mitigation.

In some embodiments, the contaminant includes perfluoroalkyl substances, polyfluoroalkyl substances, polycyclic aromatic hydrocarbons, heavy metal, or a combination thereof. The terms “perfluoroalkyl substances,” “polyfluoroalkyl substances,” and “per- and polyfluoroalkyl substances,” collectively abbreviated as “PFAS,” as used herein, refer to synthetic organofluorine chemical compounds that have multiple fluorine atoms attached to an alkyl chain. PFAS includes at least one fully fluorinated methyl or methylene carbon atom having no H, Cl, Br, or I atom attached. PFAS may include any chemical with at least one perfluorinated methyl group (—CF₃) or perfluorinated methylene group (—CF₂—). Exposure of individuals to PFAs may result in increased risk of numerous diseases or disorders, including, for example, hypercholesterolemia, ulcerative colitis, thyroid disease, testicular cancer, kidney cancer, hypertension, preeclampsia, and the like. Sources of PFAs include, for example, fluorochemical facilities that produce PFAS, carpets, furniture, waxes, flame-resistant protective wear, repellents, polishes, paints, coatings, and the like. PFAS may include, for example, perfluorinated carboxylic acids, such as pentafluoropropionic acid, perfluorobutanoic acid, perfluoropentanoic acid, perfluorohexanoic acid, perfluoroheptanoic acid, perfluorooctanoic acid, perfluorononanoic acid, perfluorodecanoic acid, perfluoroundecanoic acid, perfulorododecanoic acid, perfulorotridecanoic acid, perfluorotetradecanoic acid, and the like. Additional PFAS include, for example, perfluorobutanesulfonic acid, perfluoropentanesulfonic acid, perfluorohexanesulfonic acid, perfluoroheptanesulfonic acid, perfluorooctanesulfonic acid, perfluorononanesulfonic acid, perfluorodecanesulfonic acid, perfluorobutanesulfonamide, perfluoropentanesulfonamide, perfluorohexanesulfonamide, perfluoroheptanesulfonamide, perfluorooctanesulfonamide, perfluorobutanesulfonyl fluoride, perflourooctanesulfonyl fluoride, and the like.

The term polycyclic aromatic hydrocarbons (PAHs) refer to a hydrocarbon composed of one or more aromatic rings. Exposure of individuals to PAHs may result in increased risk of numerous diseases or disorders, including, for example, cancers, such as skin, lung, bladder, liver, and stomach cancers, cardiovascular diseases, or developmental disorders. PAHs may originate from fossil carbons, such as oil, coal, or bitumen, from combustion of organic matter, from human activity, or the like. PAHs may include, for example, anthracenes, benzenes, benzo[a]pyrenes, benzo[c]fluorenes benzo[ghi]perylenes, biphenyls, chrysenes, corannulenes, coronenes, fluorenes, naphthalenes, ovalenes, pentacenes, perylenes, phenalenes, phenanthrenes, pyrenes, tetracenes, or triphenylenes.

In some embodiments, the compositions are formulated to adsorb at least one metal. In some embodiments, the metal is a heavy metal as described elsewhere herein equal to or greater than about 5 kg/m³. In some embodiments, the metal includes ionic metal, elemental metal, or a combination thereof. In some embodiments, the metal is selected from Al, As, Ba, Be, Cd, Co, Cr, Cu, Hg, Fe, Li, Mg, Mn, Mo, Ni, Pb, Rb, Sb, Se, Sn, Sr, Tl, V, W, Zn, and any combination of the foregoing. In some embodiments, the metal is selected from As, Cd, Cu, Hg, Fe, Pb, Zn, and any combination of the foregoing. In some embodiments, the metal includes As. In some embodiments, the metal includes Cd. In some embodiments, the metal includes Cu. In some embodiments, the metal includes Hg. In some embodiments, the metal includes Fe. In some embodiments, the metal includes Pb. In some embodiments, the metal includes Zn.

In some embodiments, efficacy of the compositions is equal to or greater than about 1% to about 100%, such as 1, 2, 3, 4, 5, 10, 15, 20 25, 30, 35, 40, 45, 50, 55, 60, 65, 70, 75, 80, 85, 90, 95, 96, 97, 98, 99, or 100% or ranges including and/or spanning the aforementioned values. The term “efficacy,” as used herein, refers to the ability of the compositions to reduce or remove a contaminant from an environment or from a surface. Thus, for example 50% efficacy is an ability to remove 50% of contaminants from an environment or from a surface. The efficacy is determined by the combination of components of the compositions. In some embodiments, the combination of components includes a surfactant including decyl glucoside and disodium cocoamphodiacetate; a polymer including carboxymethylcellulose; an adsorbent including PAC; and a chelator or heavy metal chelator including trisodium ethylenediamine disuccinate, sodium gluconate, and citric acid. In some embodiments, the combination of components includes a surfactant including decyl glucoside and disodium cocoamphodiacetate; a polymer including carboxymethylcellulose; and an adsorbent including PAC. In some embodiments, the combination of components includes a surfactant including decyl glucoside and disodium cocoamphodiacetate and a polymer including carboxymethylcellulose. In some embodiments, the combination of components includes a polymer including carboxymethylcellulose and an adsorbent including PAC.

### Formulations

In some embodiments, the compositions described herein are formulated for environmental application, such as a liquid, spray, solution, coating, mist, soak, powder, or rinse. In some embodiments, the compositions described herein are formulated for personal care application, such as application to a surface, such as skin, hair, or clothing. In some embodiments, the personal care compositions are formulated as a spray, coating, solution, gel, cream, lotion, salve, paste, serum, ointment, liquid, aerosol, foam, wipe, shampoo, conditioner, soap, or cleanser.

In some embodiments, the compositions are formulated as a concentrated formulation to be diluted prior to application. In some embodiments, the compositions are formulated as a ready-to-use formulation. In some embodiments, the compositions are formulated for mass application, including in massive amounts, such as in amounts of hundreds of gallons, for example for application to an artificial turf field or playground. In some embodiments, the compositions are formulated for single use application, including in minute amounts, such as in amounts of milliliters, for example for application to hands.

For environmental application, the compositions disclosed herein may be formulated as a spray that is sprayed or misted onto a field, a playground, or other environmental area. In some embodiments, the compositions are implemented into a sprinkling system. In some embodiments, the compositions are crop-dusted over an environment. In some embodiments, the compositions are flooded or soaked over an environment. In some embodiments, a decontamination zone is prepared, within which a material is placed, and the composition is sprayed, misted, vaporized, coated, soaked, or otherwise contacted upon the material. The material may include clothing, occupational equipment, playground equipment, or the like. In some embodiments, a subject enters a decontamination zone, and the subject is contacted with the compositions to remove contaminants from skin, clothing, or other material associated with the subject. In some embodiments, the compositions are prepared as a washing machine additive, and are included in a washing cycle to remove contaminants from clothing using a washing machine. In some embodiments, the compositions are used for dust mitigation in an environment.

The term “personal care product,” as used herein, is a composition to be directly applied to the skin, mucosa, hair, nails, or other surface area of the body. Non-limiting examples of personal care products include a skincare product, a hair care product, a body care product, and a bathing product.

In some embodiments, a composition as disclosed herein can be a skincare product. Examples of skincare products include, but are not limited to face washing creams, scar smoothing products, skin irritation soothing products (oils, sprays, salves, lotions, ointments, or gels), facial cleansers, facial cleansing wipes, body cleansing wipes, anti-aging facial and/or eye creams, pore cleansing strips, pore cleansing oils, pore cleansing ointments, hair removal products (such as wax, wax strips, roll-ons, creams, gels, lotions), varnishing creams, cleansing creams, cold creams, massage creams, milky lotions, skin toning lotion, cosmetic solution, packs, makeup remover, and the like; as makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, and the like.

In some embodiments, the compositions as disclosed herein can be a hair care product. Examples of hair care products include, but are not limited to hair care cosmetics, pomades, brillantine, setting lotions, hair sticks, hair solids, hair oils, hair treatments, hair creams, hair tonics, hair liquids, hair sprays, mousse, hair gel, hair restorers, hair dyes, shampoos, permanent wave lotions, medicated shampoos, rinses, hair conditioners, hair treatments, hair packs, and the like.

In some embodiments, the compositions as disclosed herein can be a body care or bathing product. For example, the compositions can be a shaving cream, a shaving gel, a shaving lotion, an after-shave lotion, an after-shave gel, a medicated soap, sanitizing gel, a hand sanitizer, sanitizing wipes, cool nasal tissues (tissues that have lotion on them), sports cooling towels, cooling fabrics, sport shirts, eye pillows, sheets, cooling pillows, hats, diapers, adult diapers, potty-training pants and pull-ups, talcum powder, baby powder, slimming gels, a bath soap, a face soap, a hand soap, exfoliating washes, hand scrub, foot scrub, shower mists, shower sprays, a body washing soap, a body washing gel, a bath salt, a bath tablet, a bath foam, a bubble-bath concentrate, a bath oil, a bath perfume, a bath capsule, a milk bath, a bath gel, or a bath cube.

In some embodiments, the compositions as disclosed herein can be a body care product for the enhancement of massage therapy treatment. For example, the compositions can be in the form of a massage oil, massage exfoliator, massage cream, massage lotion, or massage gel.

In some embodiments, the compositions disclosed herein include: i) components as disclosed and described herein, individually or in combination; ii) a pharmaceutically acceptable carrier or pharmaceutically acceptable excipient; and iii) optionally at least one adjuvant. The terms “pharmaceutically acceptable carrier” and “pharmaceutically acceptable excipient” include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The term “adjuvant” denotes an additive which supplements, stabilizes, maintains, or enhances the intended function or effectiveness of the composition. In one embodiment, the at least one adjuvant includes one or more acceptable materials that may be used to preserve or alter the properties of the compositions disclosed herein. These materials include, but are not limited to, materials having anti-acne, anti-ageing, anti-wrinkle, antifungal, anti-inflammatory, antimicrobial, antioxidant, antiperspirant, antidandruff, anti-dermatitis, antipruritic, anti-emetic, anti-dry skin, anti-psoriatic, anti-seborrhea, anti-asthmatic, astringents, bronchodilators, biocides, chemical exfoliants, cleansers, colorants, corticosteroids, deodorants, depigmenting, depilating, emollients, epilating, analgesics, hair conditioners, hormones, humectants, light-interacting, luster-imparting, make-up removers, pH adjusters, powders, rheological modifiers, shine-imparting, skin bleaching, skin conditioning, skin protecting, tanning, UV screening vitamins, and/or wound-healing properties.

In some embodiments, the compositions disclosed herein can be in a form selected from the group consisting of liquid, including solution and suspension, solid, foamy material, emulsion, paste, gel, cream, and a combination thereof, such as a liquid containing a certain amount of solid contents.

In some embodiments, the compositions disclosed herein include an emulsifier, a skin penetration enhancer, an emollient, and any combination of the foregoing.

In some embodiments, the compositions include a skin penetration enhancer. Examples of suitable skin penetration enhancers include alcohols, fatty acids, fatty acid esters, polyols, sulphoxides, glyceryl monooleate, lauryl lactate, Dodecyl-2-(N,N-dimethyl)-amino propionate (DDAIP), N-(4-bromobenzoyl)-S,S-dimethyliminosulfurane, NexACT enhancers, 2-nonyl-1,3-dioxolane, 1-dodecylazacycloheptan-2-one, pyrrolidones, essential oil, terpenes, terpenoids, oxazolidinones, urea and the like. In some embodiments, skin penetration can be achieved by formulating the compositions disclosed herein into a nanoparticle or nanoemulsion. Such particles and emulsions are known in the art and include but are not limited to, polylactic acid particles, polylactic/glycolic acid nanoparticles, polystyrene nanoparticles, silicon dioxide nanoparticles, metallic nanoparticles, water-in-oil emulsions, oil-in-water emulsions, polymer nanoparticles and emulsions, and block copolymer nanoparticles and emulsions.

In some embodiments, the compositions include an emollient. Examples of suitable emollients include alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof. In some embodiments, the emollient can be selected from the group consisting of cetyl palmitate, stearyl palmitate, cetyl stearate, isopropyl laurate, isopropyl myristate, and isopropyl palmitate, or combinations thereof. In some embodiments, the emollient can be selected from the group consisting of octyldodecanol, lauryl, myristyl, cetyl, stearyl, and behenyl alcohol, or combinations thereof. In some embodiments, the emollient can be selected from the group consisting of eucalyptol, ceteraryl glucoside, dimethyl isosorbic polyglyceryl-3 cetyl ether, polyglyceryl-3 decyltetradecanol, propylene glycol and myristyl ether, or combinations thereof.

In some embodiments, the compositions include adjunct components conventionally found in pharmaceutical compositions. For example, the compositions may include additional compatible pharmaceutically active materials for combination therapy, such as antimicrobials, antioxidants, anti-parasitic agents, antipruritics, antifungals, antiseptic actives, biological actives, astringents, keratolytic actives, local anesthetics, anti-stinging agents, anti-reddening agents, skin soothing agents, and combinations thereof.

In some embodiments, the compositions include colorants, deodorants, fragrances, perfumes, emulsifiers, anti-foaming agents, lubricants, natural moisturizing agents, skin conditioning agents, skin protectants and skin benefit agents (e.g., aloe vera and laponite), solvents, solubilizing agents, suspending agents, wetting agents, humectants, preservatives, propellants, dyes and/or pigments, and combinations thereof. In some embodiments, the compositions may have a particularly pleasant fragrance. In some embodiments, the compositions may have a particularly pleasant texture. In some embodiments, the compositions may have a particularly pleasant soothing effect.

In some embodiments, the compositions include excipients conventionally found in compositions. In some embodiments, the excipients can include a viscosity-adjusting agent. In some embodiments, the viscosity adjusting agents can be selected from the group consisting of long chain alcohols, cellulose ethers, gums, magnesium aluminum silicate, silica, microcrystalline wax, beeswax, paraffin, and cetyl palmitate, homopolymers, and copolymers. In some embodiments, the long chain alcohols can be cetyl alcohol, stearyl alcohol, or cetearyl alcohol. In some embodiments, the cellulose ethers can be hydroxypropylmethylcellulose, hydroxyethylcellulose, hydroxypropylcellulose, or carboxymethylcellulose. In some embodiments, the gum can be xanthan gum or sclerotium gum. In a particular embodiment, the viscosity adjusting agents can include xanthan gum. In another embodiment, the viscosity adjusting agents is xanthan gum. In another embodiment, the viscosity adjusting agent is a polyalkylene oxide such as polyethylene glycol. In other embodiments, the viscosity adjusting agent is pullulan. In other embodiments, the viscosity adjusting agent is a polyvinyl halide, such as polyvinyl chloride.

In some embodiments, the compositions include colorants, deodorants, fragrances, perfumes, anti-foaming agents, lubricants, natural moisturizing agents, skin conditioning agents, skin protectants, skin benefit agents, solvents, solubilizing agents, suspending agents, wetting agents, humectants, propellants, dyes, pigments, and combinations thereof.

In some embodiments, the compositions include additional components added to enhance the odor, texture, or color of the composition. For example, fragrances may be added to enhance odor. For example, emulsifiers or inert spheres may be added to enhance texture. For example, colorants may be added to enhance color.

In some embodiments, the compositions may be applied to a body portion, such as a hand, arm, foot, leg, knee, elbow, face, head, neck, torso, and the like. The composition may be applied by any suitable means, such as rubbing, spraying, rolling, wiping, and the like, and massaged into the body portion to be treated.

In some embodiments, compositions thereof can be used in combination therapy with at least one other agent. In some embodiments, a composition thereof is administered concurrently with the administration of another agent, which may be part of the same composition of the present disclosure or a different composition. In other embodiments, a composition of the present disclosure is administered prior or subsequent to administration of another agent.

### Methods of Decontamination

Some embodiments of the present disclosure relate to methods of decontaminating an environment or a surface. An environment may include, for example, an occupation environment that is subjected to contamination, playground infields, artificial turfs, athletic fields, or the like.

In some embodiments, the methods include contacting an environment with the compositions disclosed herein. Contacting may be applied by spraying, misting, vaporizing, soaking, flooding, or coating an environment. In some embodiments, the contacting is performed by crop-dusting, use of sprinkling system, or otherwise contacting the environment. The compositions may be provided in a concentrated formulation that is diluted prior to use or may be provided in a ready-to-use formulation. In some embodiments, contacting the environment removes contaminants from the environment. In some embodiments, contacting the environment coats the contaminants, thereby reducing inhalation of contaminant particles by subjects present in the environment.

Non-limiting examples of surfaces suitable for decontamination by the methods described herein include fields, playgrounds, lawns, turfs, roadways, sidewalks, yards, construction areas, firefighting areas, clothing, shoes, socks, personal equipment, occupational equipment, athletic equipment, playground equipment, benches, tables, chairs, skin, hair, or nails of a subject.

In some embodiments, the methods include applying a composition of the present disclosure to the surface. The composition can be diluted with water or other solvent as described elsewhere herein prior to being applied. The composition may be applied by any suitable means, such as rubbing, spraying, rolling, wiping, and the like, and massaged into the body portion to be treated.

In some embodiments, the methods include removing and/or suppressing contaminants from an environment or from a surface. In some embodiments, the methods include contacting the environment or surface with any of the compositions described herein, thereby suppressing and/or removing contaminants from the environment or surface. In some embodiments, the methods include dust mitigation.

In some embodiments, the methods include absorbing contaminants from the environment or from the surface to form a composition with absorbed contaminants. In some embodiments, the contaminants and/or the absorbed contaminants include PFAS, PAHs, heavy metals, or a combination thereof. In some embodiments, the contaminants and/or the absorbed contaminants include at least one derivative of PFAS, at least one derivative of PAHs, at least one derivative of a heavy metal, and any combination of the foregoing. The term “derivative,” as herein, is a product of a chemical transformation created by one or more components of the composition.

In some embodiments, the methods include the step of removing the composition having absorbed contaminants from the surface. The composition may be removed by rinsing with water or other suitable solvent as described elsewhere herein. In some embodiments, the methods include removing contaminants from a subject. In some embodiments, the subject is an animal. In some embodiments, the subject is a human. In some embodiments, the subject is a domestical animal, such as a companion animal, including canines, felines, equines, birds, or the like.

In some embodiments, the methods reduce the contaminants from the environment or from the surface in an amount ranging from about 1% to about 100%, such as 1%, 5%, 10%, 15%, 20%, 25%, 30%, 35%, 40%, 45%, 50%, 55%, 60%, 65%, 70%, 75%, 80%, 85%, 90%, 95%, 98%, 99%, or 100% or ranges including and/or spanning the aforementioned values.

### Methods of Preparation

Some embodiments provided herein relate to methods of making the compositions and formulations described herein. In some embodiments, the methods include mixing one or more of a surfactant, a polymer, an adsorbent, or a chelator in amounts sufficient to prepare a decontaminating composition.

In some embodiments, the methods include stirring water, and adding preservatives until sufficiently mixed. In some embodiments, the methods further include adding chelators until sufficiently mixed to form a chelator solution. In some embodiments, the methods further include separately mixing a polymer in glycerin for a sufficient period of time for hydration of the polymer to form a polymer solution. For example, the polymer solution may be mixed for a period of at least 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 36 hours, 42 hours, or 48 hours. In some embodiments, the polymer solution is added to the chelator solution with constant mixing to form a combined solution. In some embodiments, one or more surfactants are added to the combined solution. In some embodiments, additional buffers, preservatives, chelators, polymers, or surfactants are added to the combined solution. In some embodiments, a surfactant is mixed into the composition. In some embodiments, an adsorbent is mixed into the composition.

In some embodiments, the pH is maintained at a desired pH to ensure that the components of the composition are not comprised, while concomitantly enabling proper mixing and preparation of the compositions. For example, the pH may be maintained or adjusted to a pH ranging from about 3 to about 8, such as 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6, 6.5, 7, 7.5, or 8 or ranges including and/or spanning the aforementioned values. The pH may be adjusted based on the target contaminant, the specific formulation, or the desired application of the compositions. For example, heavy metals may be more efficiently removed from an environment or surface at a specific pH, such that the pH may be modulated depending on which heavy metal is targeted for removal.

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### EXAMPLES

### Example 1: Method of Making the Composition

The following example demonstrates an exemplary method for making an exemplary composition for use in decontaminating an environment or surface.

Distilled water was obtained and stirred at variable speeds to control the viscosity of the compositions. Preservatives, such as potassium sorbate and sodium benzoate were added to the water under stirring. Chelators were then mixed into the solution, including sodium gluconate and trisodium ethylenediamine disuccinate. In a separate vessel, glycerin was blended with a polymer, such as carboxymethylcellulose or hydroxyethylcellulose, for a period of 48 hours to fully hydrate the polymer.

The hydrated polymer was slowly added to the chelator solution under constant mixing to avoid agglutination. A surfactant, such as decyl glucoside, and chelator, such as citric acid, were separately added. Once the pH reached between 4.0 and 4.3, the solution was protonated and ionized by pouring disodium cocoamphodiacetate into the solution and blended until completely homogenized. An adsorbent, such as protonated activated carbon (PAC), was added. The final pH of the solution was between 4.0 and 4.3.

The preparation of the compositions in this manner ensures that the disodium cocoamphodiacetate is ionized and that the protonated activated carbon maintains protonation.

### Example 2: Environmental Composition

The following example demonstrates an exemplary composition prepared by the method of Example 1.

The method of Example 1 was used to prepare a composition having one or more adsorbent, one or more chelator, one or more preservative, one or more surfactant, and one or more polymer. Specifically, the composition included the components and amounts as described in Table 1.

TABLE 1

| | Amount |
|---|---|
| Component | (wt. %) |
| Adsorbent (such as protonated activated carbon) | 0.0001-50 |
| Chelator (such as sodium gluconate, trisodium | 0.001-10 |
| ethylenediamine disuccinate, and/or citric acid) | |
| Preservative (such as potassium sorbate and/or | 0.001-3 |
| sodium benzoate) | |
| Surfactant (such as decyl glucoside and/or disodium | 0.001-50 |
| cocoamphodiacetate) | |
| Polymer (such as carboxymethylcellulose) | 0.001-7 |
| Water | balance |

The compositions were used to decontaminate an environment and a surface, and effectively removed contaminants.

### Example 3: Decontaminating Composition

The following example demonstrates an exemplary composition prepared by the method of Example 1.

The method of Example 1 was used to prepare a composition having one or more adsorbent, one or more chelator, one or more preservative, one or more surfactant, and one or more polymer. Specifically, the composition included the components and amounts as described in Table 2.

TABLE 2

| | Amount |
|---|---|
| Component | (wt. %) |
| Decyl glucoside | 16.00 |
| Disodium cocoamphodiacetate | 5.00 |
| Vegetable glycerin | 4.00 |
| Citric acid | 1.10 |
| Carboxymethylcellulose | 0.99 |
| Sodium gluconate | 0.20 |
| Trisodium ethylenediamine disuccinate | 0.20 |
| Protonated activated carbon | 0.05 |
| Potassium sorbate | 0.05 |
| Sodium benzoate | 0.05 |
| Water | balance |

A mixing apparatus fitted with a variable speed mixer was charged with 72.36 g deionized water followed by addition of 50 mg potassium sorbate, 50 mg sodium benzoate, 200 mg sodium gluconate, and 200 mg trisodium ethylenediamine disuccinate to form a mixture. In a separate vessel, 4 g vegetable glycerin and 0.99 g carboxymethylcellulose were combined and then introduced slowly into the mixture with vigorous mixing to avoid agglutination. Each of 16 g decyl glucoside, 1.1 g citric acid, and 5 g disodium cocoamphodiacetate was added to the mixture sequentially while maintaining vigorous mixing and a pH of the mixture between 4.0 and 4.3. At least one minute was allowed to pass between each addition. Protonated activated carbon (50 mg) was added to the mixture with vigorous mixing. In this manner, 100 g of the composition having a pH between 4.0 and 4.3 was obtained.

### Example 4: Methods of Decontaminating an Environment

The following example demonstrates an exemplary method of removing contaminants using the composition described in Example 3.

The composition of Example 3 was provided and formulated as a solution. The solution was provided in a five-gallon container, a 1-gallon container, or in a spray applicator container. The solution was sprayed upon artificial turf contaminated with PFAS, PAHs, and/or heavy metals. The solution remained on the artificial turn for a period of several minutes, and both cleansed the artificial turf and coated the artificial turf, thereby acting as both a cleanser and binder. The solution slowly traversed downward along the turf blades and into the backing, together with contaminants. Following 10 minutes, the artificial turf was decontaminated.

In a related method, the solution was applied to a playground infill contaminated with PFAS, PAHs, and/or heavy metals. The playground infill was a recycled tire crumb rubber. The solution remained on the playground infill for a period of several minutes, and both cleansed the playground infill and coated the playground infill, thereby acting as both a cleanser and binder. The solution slowly traversed downward along the turf blades and into the backing, together with contaminants. Following 10 minutes, the artificial turf was decontaminated.

The solution worked unexpectedly and surprisingly superior to control compositions that did not include binders that remove contaminants.

### Example 5: Methods of Decontaminating a Surface

The following example demonstrates an exemplary method of removing contaminants using the composition described in Example 3.

The composition of Example 3 was provided and formulated as a wash. The wash was provided to an individual contaminated with PFAS, PAHs, and/or heavy metals through occupation contact or contact via artificial turf or infill. The wash was rubbed onto skin of the individual for a period of several minutes, and then removed. The skin treated with the wash was cleansed of contaminants.

The wash worked unexpectedly and surprisingly superior to control compositions that did not include binders that remove contaminants.

### Example 6: Methods of Dust Mitigation

The following example demonstrates an exemplary method of mitigating dust using any of the composition described herein.

A composition was provided, including as an example, the composition of Example 3, which was formulated as a solution. The solution was provided in a five-gallon container, a 1-gallon container, or in a spray applicator container. The solution was sprayed upon artificial turf contaminated with dusts. The solution remained on the artificial turn for a period of several minutes, and suppressed dust present on the artificial turf, thereby reducing an amount of dust released from the turf into the surrounding atmosphere and environment. The compositions thereby acted as a dust suppressor or dust mitigation composition.

The solution worked unexpectedly and surprisingly superior to control compositions for dust mitigation.

Additional embodiments are set forth in the following enumerated alternatives:

1. A composition comprising: a surfactant; a polymer; an adsorbent; and a chelator.

2. The composition of alternative 1, wherein the surfactant comprises a glycoside, a cocoamphodiacetate, or a combination thereof.

3. The composition of alternative 2, wherein the glycoside comprises a C₆-30 alkyl glycoside.

4. The composition of alternative 2 or alternative 3, wherein the glycoside comprises decyl glucoside.

5. The composition of any one of alternatives 2 to 4, wherein the cocoamphodiacetate comprises disodium cocoamphodiacetate.

6. The composition of any one of alternatives 1 to 5, wherein the polymer comprises cellulose.

7. The composition of alternative 6, wherein the cellulose comprises a cellulose derivative.

8. The composition of any one of alternatives 6 to 7, wherein the cellulose comprises carboxymethylcellulose.

9. The composition of any one of alternatives 1 to 8, wherein the adsorbent comprises charcoal, activated carbon, granular activated carbon (GAC), clay minerals, bentonite, sand, or a combination thereof, optionally wherein the adsorbent is protonated.

10. The composition of any one of alternatives 1 to 9, wherein the adsorbent is protonated.

11. The composition of any one of alternatives 1 to 11, wherein the adsorbent comprises protonated activated carbon (PAC).

12. The composition of any one of alternatives 1 to 11, wherein the adsorbent is a particle having a diameter of 0.2 mm.

13. The composition of any one of alternatives 1 to 12, wherein the chelator comprises at least one heavy metal chelator.

14. The composition of any one of alternatives 1 to 13, wherein the chelator comprises an ethylenediamine, a gluconate, a polycarboxylic acid, or a combination thereof.

15. The composition of any one of alternatives 1 to 15, wherein the surfactant is present in an amount ranging from about 0.001 to about 50 weight percent.

16. The composition of any one of alternatives 1 to 15, wherein the polymer is present in an amount ranging from about 0.001 to about 10 weight percent.

17. The composition of any one of alternatives 1 to 16, wherein the adsorbent is present in an amount ranging from about 0.0001 to about 50 weight percent.

18. A composition comprising: decyl glucoside; disodium cocoamphodiacetate; carboxymethylcellulose; trisodium ethylenediamine di succinate; sodium gluconate; citric acid; sodium benzoate; potassium sorbate; protonated activated carbon; and water.

19. The composition of alternative 18, wherein the decyl glucoside is present in an amount ranging from about 0.001 to about 50 weight percent.

20. The composition of alternative 18 or alternative 19, wherein the disodium cocoamphodiacetate is present in an amount ranging from about 0.001 to about 10 weight percent.

21. The composition of any one of alternatives 18 to 20, wherein the carboxymethylcellulose is present in an amount ranging from about 0.001 to about 10 weight percent.

22. The composition of any one of alternatives 18 to 21, wherein the trisodium ethylenediamine disuccinate is present in an amount ranging from about 0.001 to about 10 weight percent.

23. The composition of any one of alternatives 18 to 22, wherein the sodium gluconate is present in an amount ranging from about 0.001 to about 10 weight percent.

24. The composition of any one of alternatives 18 to 23, wherein the citric acid is present in an amount ranging from about 0.001 to about 10 weight percent.

25. The composition of any one of alternatives 18 to 24, wherein the sodium benzoate is present in an amount ranging from about 0.001 to about 10 weight percent.

26. The composition of any one of alternatives 18 to 25, wherein the potassium sorbate is present in an amount ranging from about 0.001 to about 10 weight percent.

27. The composition of any one of alternatives 18 to 26, wherein the protonated activated carbon is present in an amount ranging from about 0.0001 to about 10 weight percent.

28. The composition of any one of alternatives 18 to 27, wherein the composition comprises: decyl glucoside present in an amount of about 16 weight percent; disodium cocoamphodiacetate present in an amount of about 5 weight percent; carboxymethylcellulose present in an amount of about 0.99 weight percent; trisodium ethylenediamine disuccinate present in an amount of about 0.2 weight percent; sodium gluconate present in an amount of about 0.2 weight percent; citric acid present in an amount of about 1.1 weight percent; sodium benzoate present in an amount of about 0.05 weight percent; potassium sorbate present in an amount of about 0.05 weight percent; protonated activated carbon present in an amount of about 0.05 weight percent; and water in balance.

29. The composition of any one of alternatives 1 to 28, wherein a pH of the composition is in the range from about 3 to about 8.

30. The composition of any one of alternatives 1 to 29, wherein the composition is configured to adsorb at least one metal.

31. The composition of alternative 30, wherein a density of the metal is equal to or greater than about 5 kg/m³.

32. The composition of alternative 31, wherein the metal is selected from Al, As, Ba, Be, Cd, Co, Cr, Cu, Hg, Fe, Li, Mg, Mn, Mo, Ni, Pb, Rb, Sb, Se, Sn, Sr, Tl, V, W, and Zn.

33. The composition of any one of alternatives 1 to 32, wherein the composition is configured to adsorb a hazardous material, including dusts or contaminants.

34, The composition of alternative 33, wherein the contaminants comprise perfluoroalkyl substances, polyfluoroalkyl substances, polycyclic aromatic hydrocarbons, heavy metals, or a combination thereof.

35. The composition of any one of alternatives 1 to 34, wherein the composition is formulated as a personal care product.

36. The composition of alternatives 35, wherein the personal care product is a cleanser, a cream, a hair conditioner, a lotion, a shampoo, a wipe, or a combination thereof.

37. A method of removing, adsorbing, remediating, and/or suppressing a hazardous material, such as dust or contaminants, comprising: applying a composition of alternative 1 or alternative 36 to a material having or suspected of having contaminants thereon; absorbing contaminants from the material to form a composition with absorbed contaminants; and removing the composition with absorbed contaminants from the material, wherein the contaminants comprise perfluoroalkyl substances, polyfluoroalkyl substances, polycyclic aromatic hydrocarbons, heavy metals, or a combination thereof.

38. The method of alternative 37, wherein the material is a surface.

39. The method of alternative 38, wherein the surface comprises skin of a subject.

40. The method of alternative 39, wherein the subject is a mammal.

41. The method of any one of alternatives 39 to 40, wherein the subject is human.

42. The method of any one of alternatives 37 to 41, wherein the heavy metal is selected from Al, As, Ba, Be, Cd, Co, Cr, Cu, Hg, Fe, Li, Mg, Mn, Mo, Ni, Pb, Rb, Sb, Se, Sn, Sr, Tl, V, W, and Zn.

Additional variations than those described herein will be apparent from this disclosure. For example, depending on the embodiment, certain acts, events, or functions of any of the compositions, systems, or methods described herein can be performed in a different sequence, can be added, merged, or left out altogether (e.g., not all described acts or events are necessary for the practice of the algorithms). Moreover, in certain embodiments, acts or events can be performed concurrently. In addition, different tasks or processes can be performed by different machines and/or computing systems that can function together.

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure. Furthermore, all references cited herein are incorporated by reference in their entirety.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

By “about” is meant a quantity, level, value, number, frequency, percentage, dimension, size, amount, weight, or length that varies by as much as 30, 25, 20, 15, 10, 9, 8, 7, 6, 5, 4, 3, 2 or 1% to a reference quantity, level, value, number, frequency, percentage, dimension, size, amount, weight, or length. When a value is preceded by the term about, the component is not intended to be limited strictly to that value, but it is intended to include amounts that vary from the value.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although system and methods have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying compositions, systems, or methods. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, sub-combinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc., are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication.

In summary, various embodiments and examples of compositions, systems, and methods have been disclosed. Although the compositions, systems, and methods have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A composition comprising:
a surfactant;
a polymer;
an adsorbent; and
a chelator.

2. The composition of claim 1, wherein the surfactant comprises a glycoside, a cocoamphodiacetate, or a combination thereof.

3. The composition of claim 2, wherein the glycoside comprises decyl glucoside.

4. The composition of claim 2, wherein the cocoamphodiacetate comprises disodium cocoamphodiacetate.

5. The composition of claim 1, wherein the polymer comprises cellulose.

6. The composition of claim 5, wherein the cellulose comprises carboxymethylcellulose.

7. The composition of claim 1, wherein the adsorbent comprises charcoal, activated carbon, granular activated carbon (GAC), clay minerals, bentonite, sand, or a combination thereof.

8. The composition of claim 1, wherein the adsorbent is a particle having a diameter of 0.2 mm.

9. The composition of claim 1, wherein the chelator comprises an ethylenediamine, a gluconate, a polycarboxylic acid, or a combination thereof.

10. The composition of claim 1, comprising:
decyl glucoside;
disodium cocoamphodiacetate;
carboxymethylcellulose;
trisodium ethylenediamine disuccinate;
sodium gluconate;
citric acid;
sodium benzoate;
potassium sorbate;
protonated activated carbon; and
water.

11. The composition of claim 18, wherein the composition comprises:
decyl glucoside present in an amount of about 16 weight percent;
disodium cocoamphodiacetate present in an amount of about 5 weight percent;
carboxymethylcellulose present in an amount of about 0.99 weight percent;
trisodium ethylenediamine disuccinate present in an amount of about 0.2 weight percent;
sodium gluconate present in an amount of about 0.2 weight percent;
citric acid present in an amount of about 1.1 weight percent;
sodium benzoate present in an amount of about 0.05 weight percent;
potassium sorbate present in an amount of about 0.05 weight percent;
protonated activated carbon present in an amount of about 0.05 weight percent; and
water in balance.

12. The composition of claim 1, wherein a pH of the composition is in the range from about 3 to about 8.

13. The composition of claim 1, wherein the composition is configured to adsorb at least one metal.

14. The composition of claim 13, wherein the metal is selected from Al, As, Ba, Be, Cd, Co, Cr, Cu, Hg, Fe, Li, Mg, Mn, Mo, Ni, Pb, Rb, Sb, Se, Sn, Sr, Tl, V, W, and Zn.

15. The composition of claim 1, wherein the composition is configured to adsorb, remediate, remove, and/or suppress a hazardous material.

16. The composition of claim 15, wherein the hazardous material comprises dust, perfluoroalkyl substances, polyfluoroalkyl substances, polycyclic aromatic hydrocarbons, heavy metals, or a combination thereof.

17. The composition of claim 1, wherein the composition is formulated as a personal care product.

18. A method of removing, remediating, adsorbing, and/or suppressing a hazardous material, the method comprising:
applying a composition of claim 1 to a material having or suspected of having a hazardous material thereon;
removing, remediating, adsorbing, and/or suppressing contaminants from the material to form a composition with absorbed contaminants; and
removing the composition with absorbed contaminants from the material,
wherein the contaminants comprise perfluoroalkyl substances, polyfluoroalkyl substances, polycyclic aromatic hydrocarbons, heavy metals, or a combination thereof.

19. The method of claim 19, wherein the material is a surface.

20. The method of claim 20, wherein the surface comprises skin of a subject.
